(19) United States
(12) Patent Application Publication
(10) Pub. No.: US 2011/0185634 A1
(43) Pub. Date: Aug. 4, 2011

(54) Device and Method for the Electrothermal-Chemical Gasification of Biomass

(76) Inventor: Michael Prestel, Sindelfingen (DE)

(21) Appl. No.: 12/991,219

(22) PCT Filed: Mar. 11, 2009
(86) PCT No.: PCT/EP09/01728

§ 371 Date: Jan. 28, 2011

(30) Foreign Application Priority Data
May 7, 2008 (EP) 10 2008 023 822.8

(51) Int. Cl. C10J 3/46; B01J 19/08; C07C 35/00

(52) U.S. Cl. 48/197 R; 422/186.04; 568/700

(57) ABSTRACT

A device for extracting fuels from biomass while adding electrical energy, comprising a gasifier for gasifying the biomass while adding electrical energy to a gas mixture, a reformer for reforming the gas mixture obtained from the gasification, a gas scrubber for scrubbing the reformed gas mixture, a catalyst for carrying out a catalytic reaction for obtaining a reaction mixture from the scrubbed gas mixture, and a separator for separating the fuel from the reaction mixture, the device further comprising devices for supplying hydrogen for hydrogenating the biomass or the gas mixture obtained from gasification and representing a closed system having a uniform internal pressure in an operating state.

The specification relates to a device and a method for the electrothermal-chemical gasification of biomass, in particular for the electrothermal-chemical gasification of biomass for extracting fuel from biomass while adding electric energy.

There are several well-known energy conversion devices and facilities such as nuclear power plants, coal-fired power plants or solar power plants and wind power plants for making energy usable to humans. In particular, an energy conversion with the aid of wind power plants, solar power plants or facilities for extracting energy from water power suffers from the drawback that they are only able to provide electric energy under specific external conditions. This means that a sufficiently strong wind, a sufficient solar irradiation or a suitable water quantity has to be available. A generation of electric energy can thus not be provided continuously or on demand but depends on external influences.

There is thus a need of being able to store in particular electric energy at the time of its generation so as to be able to use it on demand.

Several embodiments of energy storages are known in the prior art.

An object of the invention is to provide a device or a method for storing energy so as to be able to use it at a later time. The device and the method for storing energy should furthermore have a high total efficiency.

Therefore, a device for extracting fuel from biomass while adding electric energy having the following components is provided:

a gasifier for gasifying the biomass while adding electric energy to a gas mixture, wherein optionally a simultaneous hydrogenation of the heated biomass or the gas mixture obtained by the gasification can be carried out using hydrogen,
a reformer for reforming the gas mixture obtained by the gasification,
a gas scrubber for scrubbing the reformed gas mixture,
a catalytic converter for carrying out a catalytic reaction for obtaining a reaction mixture from the scrubbed gas mixture, and
a separator for separating the fuel from the reaction mixture. The device further comprises devices for supplying hydrogen for hydrogenating the biomass or the gas mixture obtained by the gasification and represents a closed system having a uniform internal pressure in an operating state.

The gas mixture obtained in the gasification is commonly also called “synthesis gas”.

In the method proposed here, the internal pressure can be automatically built up by the gasification. A uniform internal pressure in the sense of the present specification is to be understood as a system pressure of the described device which in the pressurized zones of the device has substantially the same value. Pressure differences which are inevitably required for moving the gas mixture should therefore be low relative to the predominant system pressure so that they are negligible with respect to the system pressure. The same is true in particular for the portion of the gasifier and the heat exchangers in which an enhanced pressure value can be expected because of the thermal expansion of the respective gas mixture. Incoming and outgoing volume flows can also lead to pressure increases or pressure drops in the zones of the inlets and outlets of the device to be described in further detail later which as well have a negligible value, however. The system pressure in the described device can be on the order of 10 to 200 bar, for example. In contrast, the described pressure differences are less than 0.1 bar and can thus be neglected so that the internal pressure can be regarded as uniform.

A device for extracting fuel from a synthesis gas by means of a catalytic reaction in a catalytic converter and a separation in a separator is described in the patent application filed by the applicant of the present application “Vorrichtung and Verfahren zur Treibstoffsynthese”, for example.

It should be noted that the operation of the described device and the underlying method will be illustrated in the following merely as an example with respect to alcohol which is obtained as fuel from the biomass. Instead of alcohol, other fuels such as diesel or gasoline can be generated. In this case, the material of the used catalyst potentially has to be adapted.

The optional simultaneous hydrogenation can be performed before the gasification of the biomass for hydrogenating of the biomass or for enhancing its H₂ content or later in the reforming process as was described above. Pure hydrogen as well as hydrogen containing compounds can be used. As an example, also methanol as a hydrogen containing compound and water for vapor reforming can be added for gasifying the biomass. Accordingly, the device can comprise a device for supplying hydrogen containing compounds to the biomass for its hydrogenation and/or a device for supplying water which is optional as well. A corresponding provision of hydrogen is discussed in further detail below. The supply of water can in particular be provided if the biomass is too dry so that it has to be provided with water.

For a better understanding of the processes occurring in the gasification, subsequently the reaction equations for a vapor reformation of methane and water occurring here a.) and a synthesis gas generation b.) by gasification of glucose (C₆H₁₂O₆) from biomass as well as a total reaction c.) of both reactions a.) and b.) are illustrated by way of examples. Here, the synthesis gas generation in b.) suffers from a lack of hydrogen (b: 6 H₂) which can be met by the methane produced in the vapor reformation a.) of methane, for example.

6CH₄+6H₂O→6CO+18H₂→6CH₃OH+6H₂ a.)

C₆H₁₂O₆→6CO+6H₂+(6H₂) [symbol] →6CH₃OH b.)

6CH₄+6H₂O+C₆H₁₂O₆→12CH₃OH c.)

An alternative way of providing hydrogen, for example by means of electrolysis, is subsequently discussed in further detail. Moreover, a device for generating so-called solar hydrogen for generating and supplying hydrogen can be provided.

According to an embodiment, the device comprises means for hydrogen electrolysis for supplying hydrogen in order to hydrogenate the biomass or the gas mixture obtained by the gasification. The device can thus be configured so that hydrogen for the above described hydrogenation is provided. Of course, the hydrogen can alternatively be provided by other sources such as the above-mentioned device for generating solar hydrogen. If means for hydrogen electrolysis are provided, the electric current used for carrying out the electrolysis can be obtained not only from conventional current sources but also from regenerative energy conversion facilities such as wind power plants and photovoltaic power plants.

Furthermore, the device can comprise at least one component of a group of components consisting of catalytic converters, filters, coolers, condensate separators, heat exchangers and molecular sieves.

The described device can furthermore include at least one caustic bath provided for a gas scrubbing operation for removing halogen compounds, for example. With the aid of this caustic bath, in particular fluorine and chlorine (HCl and HF) can be extracted from a respective gas mixture. As a caustic solution, sodium hydroxide NaOH can be used, for example, so that salt and water are obtained in a reaction with HCl according to the following reaction equation:

NaOH+HCl→NaCl+H₂O

Furthermore, the invention provides a method for extracting fuel such as alcohol from biomass while adding electric energy, comprising the following steps:

gasifying the biomass to a gas mixture in a gasifier,
hydrogenating the gas mixture obtained by the gasification with hydrogen and reforming the gas mixture obtained by the gasification with water vapor,
scrubbing the reformed gas mixture,
obtaining a reaction mixture from the scrubbed gas mixture by a catalytic reaction in a catalytic converter, and
separating the fuel from the reaction mixture in a separator.

The method can furthermore comprise a hydrogen electrolysis step for hydrogenating biomass or the gas mixture obtained by the gasification. This hydrogen electrolysis can take place at the same system pressure or internal pressure of the system, and alternatively a densification of the generated hydrogen is possible or necessary. The method may additionally comprise a step of the group including the following steps:

a filtering step,
a cooling step,
a condensate separation step, and
at least one step of passing through a heat exchanger.

Furthermore, the method can additionally comprise at least one of the following steps:
a) heating the gas mixture obtained by the gasification by means of electric energy and/or by local combustion of oxygen. For this purpose, a heating device which is operated electrically, for example, can be provided which additionally heats the gas mixture obtained by the gasification. Alternatively, the heating device can be operated with gas in order to additionally heat the gas mixture, or oxygen can be blown into the gas mixture for its local combustion.
b) Hydrogenating a gas mixture with hydrogen and subsequent reforming the gas mixture with water vapor. With this step, an arbitrary gas mixture can be provided with hydrogen at an arbitrary location of the device. This can be performed in the gasifier, for example, by hydrogenating the gas mixture obtained by the gasification, for example, and cause a splitting of long-chained hydrocarbon compounds (CH compounds such as coke).
c) Introducing the reformed gas mixture into a catalytic converter. For generating catalytic reactions, during and/or after the step of reforming, for example, the gas mixture can be introduced into a catalytic converter. The catalytic converter can be made of a suitable catalytic material such as cobalt or platinum which is arranged as a charge in a heat exchanger or behind it.
d) Drying the biomass before the gasification step with the aid of the gas mixture obtained by the gasification or of the reformed gas mixture by means of a counterflow heat exchanger. Here, the biomass is dried by the high temperature of the gas mixture heated by the gasification or reformation before it is supplied to the gasifier or before the gasification.
e) Using the water vapor obtained in the drying step for the reforming step. In this step, water vapor generated in the above described step of drying the biomass is used for the reformation by a water gas reaction at higher temperatures.
f) Recirculating at least a portion of the gas mixture remaining after the separation step into the gasifier. Hereby, the remaining gas mixture can be recirculated into the gasifier, for example, for performing another hydrogenation and reformation step, so that the remaining gas mixture can be subjected again to the above described method or device.
g) Separating components of the gas mixture remaining after the separation step by suitable molecular sieves. By means of these molecular sieves individual components of the remaining gas mixture such as nitrogen can be removed from the gas mixture before the remaining gas mixture is again supplied to the gasifier, as illustrated. For this purpose, the molecular sieves in the device can be arranged in a principal flow so that the portion of the remaining gas mixture recirculated in the direction of the gasifier is entirely passed through the molecular sieves. Alternatively, the molecular sieves can be arranged in an auxiliary flow so that only a partial separation occurs in the auxiliary flow and another portion of the recirculated gas mixture is directly conducted to the gasifier via a principal flow. The described device can correspondingly provide recirculation devices so that a gas mixture can be recirculated from the separator into the gasifier and supplied to the gasifier, respectively, wherein the recirculation devices have principal flow ducts and/or auxiliary flow ducts.
h) Burning a portion of the gas mixture remaining after the separation step. Alternatively or in addition to the described separation, at least a portion of the remaining gas mixture can be burnt and thus removed so that an enrichment with inert gas components such as N₂ can be avoided.

According to another embodiment, the method further comprises at least one step of a group including the following steps: a real gasification, a gas-vapor-reformation, a coke carbonization, a coke hydrogenation, a tar condensation, an electrolysis and a fuel synthesis such as an alcohol synthesis.

The device and the method described above thus enable the generation of fuel from biomass while adding electric energy. In addition to the fuel, heat is liberated. The following disclosures, as initially mentioned, only refer to alcohol as fuel for illustrative purposes. This enables the conversion of electric energy from electric current into chemical energy which is stored in the form of alcohol and can be stored relatively easily. Electric energy which is present under favorable conditions such as suitable wind conditions, sufficient solar irradiation or in another form can thus be stored in the form of alcohol.

The biomass used in this process serves as a carbon provider and can furthermore be used to enhance the efficiency. The described method allows a conversion of the carbon (C) to an alcohol such as methanol (CH₃OH) so that a conversion to CO₂ which was common in prior methods can be prevented.

The conversion and storage of electric energy in the form of alcohol allows a simple and efficient storage because alcohol can usually be obtained in the liquid state and stored in tanks. The energy stored in the alcohol can be reused or retrieved in several manners. For example, alcohol can be used as fuel or it can be converted into heat or electric current.

Apart from a simple storage, storing the alcohol in tanks enables a provision of the alcohol on demand and thus of the energy stored in it. The alcohol is available independently of external influences and can furthermore easily be transported.

Waste heat which is generated in the described electrothermal-chemical gasification of biomass for storing energy in alcohol can be made usable for heating purposes or for the generation of industrial water, for example. In this way, a relatively high total efficiency of the described method or the described device of 90-100%, for example, can be achieved using the waste heat or a so-called caloric value exploitation.

The described device for carrying out the described method can be configured as a small decentral device so that it is usable in households or single-family homes, for example. In principle, the device can be scaled arbitrarily so that larger devices or facilities can as well be realized which can be used centrally.

The described method allows an addition of the energy amounts of electric current and biomass and allows a high total efficiency by the use of the waste heat.

The use of biomass allows a diverse biomass exploitation and thus a large raw-material base. Substantially the entire carbon of the biomass can be converted into alcohol.

In this process, substantially no carbon dioxide CO₂ is generated. CO₂ is only liberated in a subsequent use of the alcohol, e.g. in the combustion of the alcohol. However, in this process only as much CO₂ is liberated as has been absorbed in the production of the biomass, e.g. in plants. Only the use of the biomass enables the storage of the electric energy of the electric current in liquid form as alcohol. The electric current used for this purpose can be spatially separated from the alcohol production. For example, wind power plants or solar power plants can be installed at favorable locations, and the generated electric current can be transported to locations for the production of alcohol at which a sufficient amount of biomass is present. The devices for the production of alcohol can of course as well be installed directly adjacent to the facilities generating electric current such as wind power plants or solar power plants, for example.

By means of the energy storage, the energy can thus be provided on demand even in times of low wind strengths or during night time. In principle, almost the entire carbon present in the biomass can be converted to alcohol, wherein substantially no CO₂ is generated. The device uses electric energy from electric current for generating or obtaining alcohol. This allows in particular a use of so-called surplus powers which in case of the described energy conversion or power generation plants usually only occur at times of low load or demand.

As already described above, the device can comprise a gasifier for the gasification of the biomass to a gas mixture or a synthesis gas. Furthermore, the device can comprise a gas scrubber for scrubbing the gas mixture or for scrubbing the gas and/or an electrolysis device, wherein in the gas scrubber among others carbon compounds can be separated from the gas mixture. The electrolysis device uses electric current for producing hydrogen by means of electrolysis. Furthermore, alcohol, e.g. methanol (CH₃OH), can be generated from the gas mixture, e.g. the synthesis gas and hydrogen (H₂), in a separator of the device by means of an alcohol synthesis. The alcohol is correspondingly extracted and can be stored in tanks. The waste heat of the described device is usable for heating purposes or the generation of industrial water, for example, or it can be extracted in suitable means as process heat.

As disclosed above, the described device for extracting alcohol can use one or several (partial) methods from a group of methods. This group comprises an ideal gasification, a real gasification, a gas-vapor-reformation, a coke carbonization, a coke hydrogenation, a tar condensation, an electrolysis and a methanol synthesis.

The above described method for extracting alcohol can furthermore comprise the following steps: heating the biomass by means of electric current or gasifying the biomass to a gas mixture and cracking of carbon-hydrogen compounds (CH compounds) which are included in the gas mixture by means of the so-called steam reforming process or gas-vapor reforming process. The gas mixture generated and simultaneously heated in the gasification can be passed for a heat recirculation through counterflow heat exchangers and thus be used for heating the generated gas mixture as well as the biomass. In this manner, only energy losses have to be compensated by electric energy of the used electric current, and otherwise an energy supply can be realized by recirculating the heat.

Furthermore, a so-called intermittent operation can be used for burning coke with oxygen O₂ and water H₂O. The gas mixture or the synthesis gas obtained in the described method includes carbon monoxide (CO), carbon dioxide (CO₂) and hydrogen (H₂). By means of a subsequent methanol synthesis, alcohol can be obtained from the gas mixture or synthesis gas.

The described gas mixture (if not otherwise specified) is to be regarded as the gasified gas mixture generated in the gasification, the components or the composition of which can vary by respective reactions in the individual steps or in the use of the individual (partial) methods.

The described method can include a multi-stage gas processing consisting of several steps. In the gasification of the biomass, a heating by means of the electric energy of the electric current as well as an additional heating of the gas mixture by means of counterflow heat exchangers occur. Furthermore, the gas processing can include, as mentioned above, a gas-water vapor-reformation, a coke carbonization with oxygen (O₂) and a tar-condensate-water vapor-reformation. A separation of potentially generated ashes which is generated in particular in the gasification of the biomass can be carried out by means of a pre-separation in an ash tray having grates. The device can furthermore include electrostatic filters configured for burning ashes. Furthermore, a use of fine-tissue filters is possible. In order to avoid a contamination or clogging of filters, so-called regeneration cycles can be provided in a control of the device. Furthermore, a so-called purge cycle can be used for the alcohol synthesis such as the methanol synthesis.

By means of the described method, a potential separation of long-chained carbon compounds, in particular of hydrocarbon compounds from the biomass, such as tar precipitations, can be avoided because they are present in a gaseous state at high temperatures. They only condense in the cooling process and can lead to congestions. However, by suitable recirculation devices, the carbon compounds or the tar containing substances of the gas mixture can pass the device or portions of the device several times until the tar or tar residues have completely been degraded. This can be achieved by a so-called cracking or splitting the long-chained carbon compounds.

The above described regeneration cycle can for instance include a burn-off of the device by a short-time heating of the entire system or the entire device or of portions of the device. The described separation of the ashes can occur by means of electrostatic filters, for example, which can be cleaned by regeneration. Furthermore, a burn-off of filter surfaces in ash boxes is possible. Electrostatic filters, in contrast to fine filters such as fine-tissue filters, do not require any service apart from emptying the ash boxes. Of course, a use of the fine filters or fine-tissue filters which can be cleaned or changed on demand is also possible.

The catalyst used in the device allows a long lifetime if biomass with a low sulfur content is used. However, if biomass having a high sulfur content is used, a cyclic replacement of the catalyst may be necessary. Furthermore, a sulfur filter in the form of a desulfurization stage can be used. The latter can be provided in the form of a zinc oxide layer (ZnO) on a suitable carrier. In the zinc oxide layer, for example H₂S (hydrogen sulfide) can be converted by means of ZnO to ZnS (zinc sulfide) and H₂O (water), wherein the described reaction can occur in a temperature range between 200 and 400° C., for example. A condensate removal which may be required in the device can be carried out by means of a separation with water and condensate, for example.

In principle, any organic materials can be used as biomass. These include in particular wood, wood chips, pellets as well as domestic trash, paper, cardboard, straw, grass and green waste. Algae, plankton and agricultural wastes can also be used. PVC-free plastics or shredder waste can also be used as biomass. Here, the biomass can be provided in solid form or also in liquid form. Liquid biomass is for instance known under the name “bio slurry” and offers the advantage of a considerably reduced volume with respect to biomass in solid form. The above list only serves as an example and should not be regarded as complete, however.

The efficiency of the described method or the described device strongly depends on the used biomass. In small and decentral devices, higher-rate biomass can therefore be used in order to provide a sufficient efficiency, for example, whereas in large devices almost any biomass even with a lower efficiency can be used.

As described above, the device can be scaled differently so that different performance stages can be achieved. The lower the direct conversion efficiency, the more economical are small decentral systems with heat exploitation. The higher the efficiency, the more practical are large devices.

At this stage, explicit reference should be made again to the initial remark that the generation of alcohol as fuel is described merely as an example and that other fuels apart from alcohol can be generated with the described method and the described device as well.

Other advantages and modifications of the invention will be understood with reference to the specification and the accompanying drawings.

It should be understood that the above mentioned features and the features to be explained below can not only be used in the respective indicated combination but also in other combinations or individually without leaving the scope of the present invention.

The invention is schematically illustrated in the drawings with respect to embodiments and will be described in further detail below with reference to the drawings.

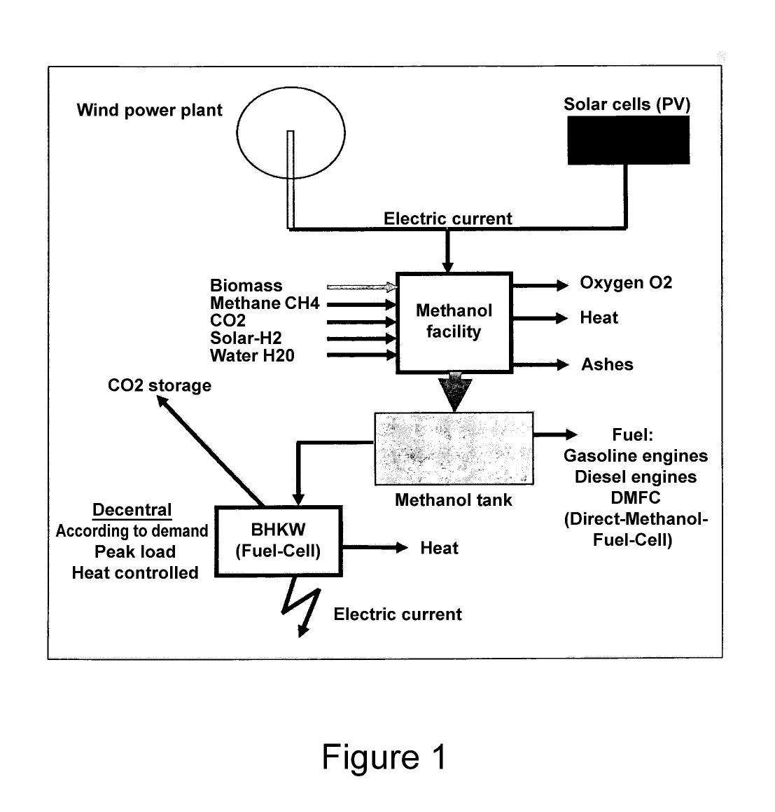

FIG. 1 shows a schematic illustration of a system for extracting alcohol from electric current and biomass.

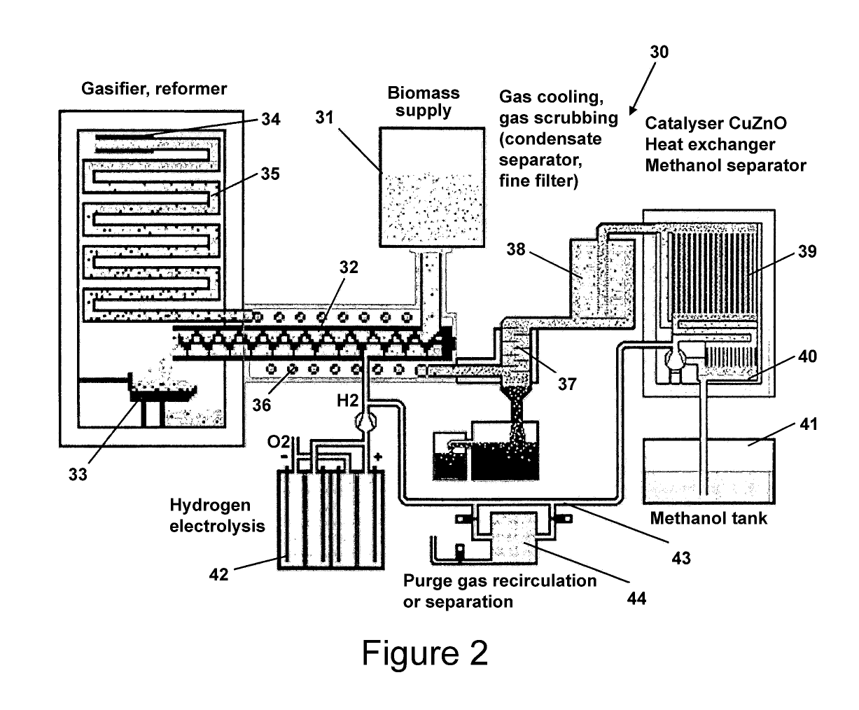

FIG. 2 shows a schematic illustration of a device for extracting alcohol from biomass while adding electric energy from electric current.

FIG. 1 shows a schematic illustration of a system for extracting fuel from biomass with the aid of electric current using alcohol and methanol, respectively, as an example. Thus, the system comprises a device 300 for extracting methanol into which the biomass for generating the methanol is introduced and which is described in detail in the following FIG. 2. Aside from the biomass, additional methane and/or H₂ for hydrogenating the biomass can optionally be introduced. In this case, the H₂ can be generated either by hydrogen electrolysis (if corresponding devices are provided for this purpose) or be supplied by a corresponding facility (not illustrated) in the form of solar hydrogen (solar-H₂), for example. Apart from the biomass, CO₂ can moreover be introduced instead of or in addition to the biomass for the operation of the device 300. Moreover, water can optionally be used for gasifying the biomass and for a vapor reformation of methane and water, respectively. The electric current used for the operation of the device 300 can be derived from conventional sources or from regenerative sources such as wind power plants 11 and/or photovoltaic power plants 12. The device 300 produces inorganic components of the supplied materials in the form of ash as well as heat which is generated in the chemical reactions occurring in the device and the desired fuel in the form of methanol. If the device 300 provides devices for hydrogen electrolysis, oxygen can also be produced. The generated methanol is extracted from the device 300 and introduced into a tank 13 for storage purposes from which it can be withdrawn on demand as fuel for multiple purposes. Apart from a merely illustrative use in correspondingly adapted gasoline and diesel engines, a use in corresponding fuel cells such as direct methanol fuel cells (DMFC) as well as in a cogeneration unit 14 is feasible. In the conversion to electric current, heat and CO₂ and potentially water are also generated at these stages.

FIG. 2 shows a device for extracting alcohol from biomass while adding electric energy. For this purpose, the device 30 comprises a biomass container 31 for storing biomass. From the container 31, the biomass is transported via a feeder 32 to a gasifier 33 where the biomass is gasified to a gas mixture while adding electric energy. For this purpose, the gasifier is heated by means of the electric energy, and the biomass is burnt or gasified by pyrolysis. Any ashes can be removed by suitable devices (not illustrated). The gasified gas mixture generated in the gasification ascends in a housing of the gasifier 33. At the upper end of the housing of the gasifier 33, a heating device 34 is arranged in which the gas mixture generated in the gasification is further heated electrically and introduced from the heating device 34 into a heat exchanger 35. Alternatively, the heating device 34 can include an afterheating with oxygen (not illustrated) so that oxygen is supplied and burnt for generating heat. The heat exchanger 35 comprises a reformer for reforming the gas mixture generated in the gasification and conducts the reformed gas mixture within the housing of the gasifier 33 in a direction opposite to the ascending gasified gas mixture and conducts the reformed gas mixture out of the housing of the gasifier 33. The heat exchanger 35 is designed so that it conducts the reformed gas mixture in a direction opposite to the ascending gasified gas mixture so that it heats the ascending gasified gas mixture with the help of the reformed gas mixture which has been further heated in the reforming process.

Furthermore, a charge with a catalytic function can be provided in the heat exchanger 35 and in corresponding conduits of the heat exchanger 35, respectively, so that a catalytic converter can additionally be provided. This charge can be cobalt, platinum or other suitable catalytically acting materials, for example. After the reformed gas mixture has been output, it is conducted out of the heat exchanger 35 and into another second heat exchanger 36 arranged along the feeder 32 and providing for a heat exchange between the reformed gas mixture and the biomass transported in the feeder.

The reformed gas mixture is subsequently conducted into a gas scrubber 37 for scrubbing the reformed gas mixture. In this process, carbon containing compounds, in particular hydrocarbon containing compounds (CH) such as tar are extracted from the reformed gas mixture. These extracted carbon containing compounds can be resupplied by suitable devices to the biomass and pass the gasifier 33, the heating device 34 and the heat exchanger 35 having a reformer in another passage and they can thereby be removed. In this process, the usually long-chained carbon containing compounds are split by the so-called cracking. The device for resupplying carbon containing compounds is not shown in FIG. 2, however. The scrubbed gas mixture generated in the gas scrubber 37 can optionally be filtered in a filter 38, as illustrated, which can be configured as a fine-tissue filter or an electrostatic filter, for example. The gas mixture filtered or scrubbed in this process is now conducted to a catalytic converter 39 for extracting alcohol from the scrubbed or filtered gas mixture by a catalytic reaction. Subsequently, the alcohol is separated in a separator 40 and extracted into a tank 41. Because in the catalytic reaction in a catalytic converter only a portion of the gas mixture reacts, the device can be configured so that the gas mixture remaining after the separator 40 passes the catalytic converter 39 several times in order to react remaining residues or components of the gas mixture as well. For this purpose, the remaining gas mixture is again supplied to the catalytic converter 39 after passing the separator 40. The remaining gas mixture is admixed to the filtered or scrubbed gas mixture supplied by the gas scrubber 37 or the filter 38, and they are both introduced into the catalytic converter 39.

Furthermore, the device 30 comprises a device for hydrogen electrolysis 42 for providing hydrogen. The hydrogen is introduced into the feeder 32 and/or the gasifier 33 and/or in the portion of the separator 40, for example. Moreover, the device 30 is configured so that the entire device 30 represents a closed system to which a uniform internal pressure can be applied. The internal pressure can be generated by gasifying the biomass, for example, and in this way provide the system pressure which is in particular required for the catalytic converter.

A uniform internal pressure in the sense of the present specification is to be understood as a system pressure of the described device which in the pressurized zones of the device has substantially the same value at any given time, even though it is temporarily variable. Local pressure differences which are inevitably required for moving the gas mixture should therefore be low relative to the predominant system pressure so that they are negligible with respect to the system pressure. The same is true in particular for the portion of the gasifier and the heat exchangers in which an enhanced pressure value can be expected because of the thermal expansion of the respective gas mixture. Incoming and outgoing volume flows can also lead to pressure increases or pressure drops in the inlet and outlet zones of the device which as well have a negligible value, however. The system pressure in the described device can be on the order of 10 to 200 bar, for example. In contrast, the described pressure differences are less than 0.1 bar and can thus be neglected. The internal pressure can in contrast vary in time within the described limits of 10 bar to 200 bar. Nevertheless, at any given time the same local internal pressure predominates in the entire device if the described local pressure differences are neglected so that the internal pressure can be regarded as uniform.

A configuration of the entire device as a closed system having a uniform internal pressure in particular allows a direct feeding of the biomass from the biomass container 31 because it is integrated into the entire system as well and is subject to the internal pressure as well. A connection of the biomass container 31 via a pressure valve is thus not required.

The biomass container 31 can be configured so that it contains or stores a certain amount of the biomass for an operation of the facility of several hours or an entire day, for example. Only when the container is empty, the internal pressure of the system is lowered to the atmospheric pressure, and the filling of the biomass container 31 can again be performed. Subsequently, the gasification is restarted, whereby the required system pressure or internal pressure of the system is automatically established.

By means of a so-called “purge gas recirculation” 43, at least a portion of the remaining gas mixture can be recirculated into the gasifier 33 for gasification after the illustrated alcohol separation in the separator 40. In this manner, gases or components of the remaining gas mixture such as methane can again be converted to CO and H₂ in the reformer. An enrichment of nitrogen and other inert gases or gas components can be avoided by a cyclical or continuous partial separation from the recirculated remaining gas mixture (so-called circulation gas or purge gas). From this so-called “purge gas” either the undesired portion or the corresponding molecules can be separated by molecular sieves 44 or the purge gas is burnt directly. The heat generated in this process can be used for the described method or the described device.

1-13. (canceled)

14. Device for extracting fuel from biomass while adding electric energy from electric current, comprising:
a gasifier for gasifying the biomass while adding electric energy to a gas mixture, wherein the gasifier is heated by means of electric energy and the biomass is gassed by pyrolysis;
a reformer for reforming the gas mixture obtained by the gasification;
a gas scrubber for scrubbing the reformed gas mixture;
a catalytic converter for carrying out a catalytic reaction for obtaining a reaction mixture from the scrubbed gas mixture;
a separator for separating the fuel from the reaction mixture; and
the device further comprising devices for supplying hydrogen for hydrogenating the biomass or the gas mixture obtained by the gasification, and representing a closed system having a uniform internal pressure in an operating state.

15. Device according to claim 14, and further comprising a device for supplying hydrogen containing compounds to the biomass for hydrogenating the same.

16. Device according to claim 14, and further comprising means for carrying out a hydrogen electrolysis for providing hydrogen for hydrogenating the biomass or the gas mixture obtained by the gasification.

17. Device according to claim 14, and further comprising at least one component of a group of components consisting of catalytic converters), filters, coolers, condensate separators, heat exchangers and molecular sieves.

18. Device according to claim 14, and further comprising recirculation devices so that a gas mixture from the separator is recirculated into the gasifier or supplied to the gasifier, wherein the recirculation devices have principal flow ducts and/or auxiliary flow ducts.

19. Device according to claim 14, and further comprising a device for supplying CO₂.

20. Device according to claim 14, wherein alcohol is obtained as fuel from the biomass.

21. Method for extracting fuel from biomass while adding electric energy from electric current, comprising:
gasifying the biomass to a gas mixture in a gasifier;
providing hydrogen and/or hydrogen containing compounds;
hydrogenating and reforming the gas mixture obtained by the gasification;
scrubbing the reformed gas mixture;
obtaining a reaction mixture from the scrubbed gas mixture by a catalytic reaction; and
separating the fuel from the reaction mixture in a separator.

22. Method according to claim 21, and further comprising the step of hydrogen electrolysis for hydrogenating biomass or the gas mixture obtained by the gasification.

23. Method according to claim 21, and further comprising at least one step of the group including the following steps:
a filtering step;
a cooling step;
a condensate separation step; and
at least one step of passing through a heat exchanger.

24. Method according to claim 23, wherein the group of steps further includes at least one of the following steps:
eating the gas mixture obtained by the gasification by means of electric energy and/or by local combustion of oxygen;
hydrogenating a gas mixture with hydrogen and subsequent reforming the gas mixture with water vapor;
introducing the reformed gas mixture into a catalytic converter;
drying the biomass before the gasification step with the aid of the gas mixture obtained by the gasification or of the reformed gas mixture by means of a counterflow heat exchanger;
using the water vapor obtained in the drying step for the reforming step;
recirculating at least a portion of the gas mixture remaining after the separation step into the gasifier;
separating components of the gas mixture remaining after the separation step by suitable molecular sieves; and
burning at least a portion of the gas mixture remaining after the separation step.

25. Method according to claim 21, and further comprising at least one step of a group including the following steps: a real gasification, a gas-vapor-reformation, a coke carbonization, a coke hydrogenation, a tar condensation, an electrolysis and a fuel synthesis.

26. Method according to claim 24, wherein alcohol is obtained as fuel from the biomass.
